(19) United States
(12) Patent Application Publication
(10) Pub. No.: US 2024/0124804 A1
(43) Pub. Date: Apr. 18, 2024

(54) ALPHA-AMYLASE MUTANT

(71) Applicant: KAO CORPORATION, Tokyo (JP)

(72) Inventors: Mao SHAKU, Wakayama-Shi, Wakayama (JP); Takahiro HIOKI, Wakayama-Shi, Wakayama (JP); Akihito KAWAHARA, Wakayama-Shi, Wakayama (JP)

(73) Assignee: KAO CORPORATION, Tokyo (JP)

(21) Appl. No.: 18/023,778

(22) PCT Filed: Jul. 20, 2021
(86) PCT No.: PCT/JP2021/027224

§ 371 Date: Dec. 20, 2023

(30) Foreign Application Priority Data
Aug. 31, 2020 (JP) 2020-146078

(51) Int. Cl. C11D 3/386; C12N 9/28; C12N 15/75

(52) U.S. Cl. CPC C11D 3/38618; C12N 9/2417; C12N 15/75; C12Y 302/01001; C11D 2111/12; C11D 2111/14; C12N 2800/101

(57) ABSTRACT

Provided is an α-amylase which exhibits high amylolytic activity at low temperatures. An α-amylase mutant, wherein an amino acid residue at a position of the following (a) and (b); (c); or (a), (b), and (c), in the amino acid sequence of SEQ ID NO: 2 or an amino acid sequence having at least 90% identity thereto is substituted with another amino acid residue: (a) position 303 of the amino acid sequence of SEQ ID NO: 2 or a position corresponding thereto; (b) one or more positions selected from the group consisting of position 295 and position 296 of the amino acid sequence of SEQ ID NO: 2 and positions corresponding thereto; or (c) position 331 of the amino acid sequence of SEQ ID NO: 2 or a position corresponding thereto.

### FIELD OF THE INVENTION

The present invention relates to an α-amylase mutant.

### BACKGROUND OF THE INVENTION

α-Amylases are used in a wide range of industries, such as starch, brewing, textiles, pharmaceuticals, and food, are known to have suitability for containing in cleaning agents, and are incorporated into dishwashing cleaning agents for automatic dishwashers and clothing cleaning agents as components removing starch stains.

Known α-amylases useful for cleaning agents are α-amylases derived from Bacillus bacteria, such as Bacillus sp. KSM-1378 (FERM BP-3048) strain-derived α-amylase AP1378 (Patent Literature 1), Termamyl and Duramyl (registered trademarks), which are Bacillus licheniformis-derived α-amylases, Bacillus sp. DSM12649 strain-derived α-amylase AA560 (Patent Literature 2), and Bacillus sp. SP722 strain-derived α-amylase SP722 (SEQ ID NO: 4 of Patent Literature 3). Also known are Cytophaga-derived α-amylase CspAmy2 (Patent Literature 4), and the like.

In recent years, from the viewpoint of environmental protection and cleaning cost reduction, it is important to reduce temperatures in dishwashing and laundry washing, particularly in laundry washing in laundries. In addition, shortening of the cleaning time is also desired. However, optimal temperatures of most enzymes, including amylases, are higher than temperatures generally set for low-temperature cleaning. For this reason, it is difficult to completely remove many starch stains.

Therefore, it is important to find α-amylases maintaining cleaning performance and amylolytic activity even at low temperatures, and having a high stain removal effect.

It has been reported that the cleaning performance of α-amylases at low temperatures is inversely correlated with amylase binding to starch (starch absorption), and that amylases with low starch absorption have high cleaning performance at low temperatures (Patent Literature 5). Patent Literature 5 discloses that starch absorption can be reduced by introducing mutations into a known starch-binding residue and its adjacent residue.

Mutants using CspAmy2 as a parent enzyme have been created (Patent Literatures 4 and 6); however, mutants with sufficiently enhanced amylolytic activity at low temperatures have not been reported.
[Patent Literature 1] WO 94/26881
[Patent Literature 2] WO 00/60060
[Patent Literature 3] WO 06/002643
[Patent Literature 4] WO 2014/164777
[Patent Literature 5] JP-A-2014-520517
[Patent Literature 6] JP-A-2019-500058

### SUMMARY OF THE INVENTION

The present invention relates to the following:
(1) An α-amylase mutant in which an amino acid residue at a position of the following (a) and (b); (c); or (a), (b), and (c), in the amino acid sequence of SEQ ID NO: 2 or an amino acid sequence having at least 90% identity thereto is substituted with another amino acid residue:
(a) position 303 of the amino acid sequence of SEQ ID NO: 2 or a position corresponding thereto;
(b) one or more positions selected from the group consisting of position 295 and position 296 of the amino acid sequence of SEQ ID NO: 2 and positions corresponding thereto; or
(c) position 331 of the amino acid sequence of SEQ ID NO: 2 or a position corresponding thereto.
(2) A polynucleotide encoding the mutant according to (1).
(3) A vector or DNA fragment comprising the polynucleotide according to (2).
(4) A transformed cell comprising the vector or DNA fragment according to (3).
(5) A cleaning composition comprising the mutant according to (1).

### BRIEF DESCRIPTION OF DRAWINGS

FIG. 1 shows the relative amylolytic activity of each amylase at 20° C.

FIG. 2 shows the relative amylolytic activity of each amylase at 20° C.

FIG. 3 shows the relative amylolytic activity of each amylase at 20° C.

FIG. 4 shows the relative amylolytic activity of each amylase at 20° C.

FIG. 5 shows the relative amylolytic activity of each amylase at 20° C.

FIG. 6 shows the detergency of each amylase at 20° C.

### DETAILED DESCRIPTION OF THE INVENTION

In the present specification, “amylase” (EC3.2.1.1; α-D-(1→4)-glucan glucanohydrolase) refers to a group of enzymes that catalyze the hydrolysis of starch and other linear or branched 1,4-glycoside oligosaccharides or polysaccharides. The α-amylase activity can be determined by measuring the amount of reducing ends produced by the enzymatic degradation of starch. The determination method is not limited thereto; for example, the α-amylase activity can also be determined by measuring the release of dye by the enzymatic degradation of dye-crosslinked starch, such as Phadebas (Soininen, K., M. Ceska, and H. Adlercreutz. “Comparison between a new chromogenic α-amylase test (Phadebas) and the Wohlgemuth amyloclastic method in urine.” Scandinavian journal of clinical and laboratory investigation 30.3 (1972): 291-297.).

In the present specification, the identity of amino acid sequences or nucleotide sequences is calculated by the Lipman-Pearson method (Science, 1985, 227: 1435-1441). Specifically, the identity is calculated by analysis using a homology analysis (Search homology) program of genetic information processing software GENETYX Ver. 12 at a unit size to compare (ktup) of 2.

The “amino acid residues” herein refer to 20 amino acid residues constituting protein, i.e., alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V).

Amino acid substitutions may herein be denoted as [original amino acid, position, substituted amino acid] according to the officially recognized IUPAC one-letter amino acid abbreviation. For example, substitution of tyrosine at position 303 with asparagine is expressed as “Y303N.”

Mutants including multiple substitutions are expressed by using the addition symbol (“+”). For example, “Y295E+Y303N” represents substitution of tyrosine at position 295 with glutamic acid and substitution of tyrosine at position 303 with asparagine, respectively.

The “operable linkage” between a regulatory region, such as a promoter, and a gene herein means that the gene and the regulatory region are linked so that the gene can be expressed under the control of the regulatory region. Procedures for the “operable linkage” between the gene and the regulatory region are well known to a person skilled in the art.

The “upstream” and “downstream” relating to a gene herein refer to the upstream and downstream in the transcription direction of the gene. For example, “a gene located downstream of a promoter” means that the gene is present on the 3′ side of the promoter in the DNA sense strand, and the upstream of a gene means a region on the 5′ side of the gene in the DNA sense strand.

The term “original” used herein for a function, property, or trait of a cell is used to indicate that the function, property, or trait is inherent in the cell. In contrast, the term “foreign” is used to describe a function, property, or trait that is introduced from outside the cell, rather than being inherent in the cell. For example, a “foreign” gene or polynucleotide is a gene or polynucleotide introduced into the cell from outside. The foreign gene or polynucleotide may be derived from an organism of the same species as the cell into which the foreign gene or polynucleotide is introduced, or from an organism of a different species (i.e., heterologous gene or polynucleotide).

For the development of α-amylases which exhibit higher detergency at low temperatures in comparison with the existing α-amylases for a cleaning agent, two points, i.e., maintenance of amylolytic activity at low temperatures and low starch absorption, are considered to be particularly important; however, it is not easy to maintain amylolytic activity at low temperatures. Conventionally, none of existing amylases for a cleaning agent sufficiently maintains amylolytic activity at low temperatures, despite the fact that the strength of activity has been considered as part of the screening criteria.

Therefore, in order to develop amylases exhibiting higher detergency at low temperatures in comparison with the existing amylases for a cleaning agent, it is important to newly find α-amylases holding high amylolytic activity at low temperatures. However, there is no sequence index for screening α-amylases holding higher amylolytic activity at low temperatures than the existing α-amylases, and it is not easy to search for such α-amylases.

The present invention relates to the provision of an α-amylase mutant with enhanced amylolytic activity at low temperatures.

As a result of measuring, at a low temperature of 20° C., the amylolytic activity of Bacillus koreensis-derived α-amylase found from existing α-amylases derived from Bacillus bacteria and estimated α-amylase sequences included in the NCBI protein sequence database, the present inventors found that the Bacillus koreensis-derived α-amylase has excellent amylolytic activity, and that specific mutants of the Bacillus koreensis-derived α-amylase have high amylolytic activity at low temperatures. Thus, the present invention was completed.

The α-amylase mutant of the present invention has enhanced amylolytic activity at low temperatures in comparison with the parent α-amylase, and allows excellent starch stain removal even when used for low-temperature cleaning.

### <α-Amylase Mutant>

The α-amylase mutant of the present invention (referred to as “the mutant of the present invention”) is an α-amylase mutant in which an amino acid residue at a position of the following 1) and/or 2) in the amino acid sequence of SEQ ID NO: 2 or an amino acid sequence having at least 90% identity thereto is substituted with another amino acid residue:
1) (a) position 303 of the amino acid sequence of SEQ ID NO: 2 or a position corresponding thereto, and (b) one or more positions selected from the group consisting of position 295, position 296 of the amino acid sequence of SEQ ID NO: 2 and positions corresponding thereto; and/or
2) (c) position 331 of the amino acid sequence of SEQ ID NO: 2 or a position corresponding thereto.
   The phrase “1) and/or 2)” as mentioned herein means 1) alone, 2) alone, or 1) and 2).

That is, the mutant of the present invention is an α-amylase mutant in which an amino acid residue at a position of the following (a) and (b); (c); or (a), (b), and (c), in the amino acid sequence of SEQ ID NO: 2 or an amino acid sequence having at least 90% identity thereto is substituted with another amino acid residue:
(a) position 303 of the amino acid sequence of SEQ ID NO: 2 or a position corresponding thereto;
(b) one or more positions selected from the group consisting of position 295 and position 296 of the amino acid sequence of SEQ ID NO: 2 and positions corresponding thereto; or
(c) position 331 of the amino acid sequence of SEQ ID NO: 2 or a position corresponding thereto.

Preferably, the mutant of the present invention is an α-amylase mutant in which an amino acid residue at a position of the above (a) and (b); (c); or (a), (b), and (c), in the amino acid sequence of SEQ ID NO: 2 or an amino acid sequence having at least 90% identity thereto is substituted with another amino acid residue, wherein the substitution of the amino acid residue at the position of (a) is substitution to A, R, N, D, C, Q, E, G, H, I, L, K, M, S, T, or V; the substitution of the amino acid residue at the position of (b) is one or both of (b-1) substitution of an amino acid residue at position 295 or a position corresponding thereto to Q, E, G, H, L, M, S, T, or V, and (b-2) substitution of an amino acid residue at position 296 or a position corresponding thereto to Y; and the substitution of the amino acid residue at the position of (c) is substitution to S.

That is, the “mutant” refers to a polypeptide having α-amylase activity in which, among the amino acids constituting the parent α-amylase, an amino acid residue at the above predetermined position is substituted. The substitution of an amino acid residue at such a predetermined position is intended to enhance amylolytic activity at low temperatures. Therefore, the mutant has enhanced amylolytic activity at low temperatures in comparison with the parent α-amylase.

The “corresponding position” on the amino acid sequence can be determined by aligning the target sequence and the reference sequence (the amino acid sequence of SEQ ID NO: 2 in the present invention) so as to give maximum homology. Alignment of the amino acid sequences can be performed using known algorithms, the procedure of which is known to a person skilled in the art. For example, alignment can be performed by using the Clustal W multiple alignment program (Thompson, J. D. et al., 1994, Nucleic Acids Res. 22: 4673-4680) with default settings. Alternatively, Clustal W2 and Clustal omega, which are revised versions of Clustal W, can also be used. Clustal W, Clustal W2, and Clustal omega are available on the website of, for example, the European Bioinformatics Institute (EBI [www.ebi.ac.uk/index.html]) or the Japan DNA Data Bank operated by National Institute of Genetics (DDBJ [www.ddbj.nig.ac.jp/searches-j.html]). A position in the target sequence that is aligned to any position in the reference sequence by the above alignment is regarded as the “position corresponding” to the any position.

A person skilled in the art can further refine the alignment of the amino acid sequences obtained above to optimize them. Such optimal alignment is preferably determined in consideration of the similarity of amino acid sequences, the frequency of inserted gaps, and the like. The similarity of amino acid sequences as mentioned herein refers to, when two amino acid sequences are aligned, the ratio (%) of the number of positions where the same or similar amino acid residues are present in both sequences to the number of full-length amino acid residues. Similar amino acid residues refer to, among the 20 amino acids constituting protein, amino acid residues which have similar properties to each other in terms of polarity and charge, and which undergo so-called conservative substitution. A group consisting of such similar amino acid residues is well known to a person skilled in the art, and examples include, but are not limited to, arginine and lysine or glutamine; glutamic acid and aspartic acid or glutamine; serine and threonine or alanine; glutamine and asparagine or arginine; leucine and isoleucine; and the like.

The α-amylase consisting of the amino acid sequence of SEQ ID NO: 2 or an amino acid sequence having at least 90% identity thereto is the “parent α-amylase” of the mutant of the present invention. The “parent α-amylase” refers to a reference α-amylase that becomes the mutant of the present invention when a specific mutation is made to its amino acid residue.

In the present invention, the protein consisting of the amino acid sequence of SEQ ID NO: 2 is a protein estimated as an α-amylase in the NCBI protein sequence database. Specifically, the protein consisting of the amino acid sequence of SEQ ID NO: 2 is registered as accession number KOO43440.1 (referred to as “BkoAmy” in the present invention) in this database.

Examples of α-amylases consisting of an amino acid sequence having at least 90% identity to the amino acid sequence of SEQ ID NO: 2 include α-amylases consisting of an amino acid sequence having at least 90% identity, specifically 90% or more, preferably 95% or more, more preferably 96% or more, even more preferably 97% or more, even more preferably 98% or more, and even more preferably 99% or more identity to the amino acid sequence of SEQ ID NO: 2.

The amino acid sequences having at least 90% identity include amino acid sequences having deletion, insertion, substitution, or addition of one or several amino acids. Examples of the “amino acid sequences having deletion, insertion, substitution, or addition of one or several amino acids” include amino acid sequences having deletion, insertion, substitution, or addition of 1 or more and 30 or less, preferably 20 or less, more preferably 10 or less, and even more preferably 5 or less amino acids.

The parent α-amylase is preferably one having tyrosine at position 303 of the amino acid sequence of SEQ ID NO: 2 or a position corresponding thereto, preferably one having tyrosine at position 295 of the amino acid sequence of SEQ ID NO: 2 or a position corresponding thereto, preferably one having alanine at position 296 of the amino acid sequence of SEQ ID NO: 2 or a position corresponding thereto, and preferably one having threonine at position 331 of the amino acid sequence of SEQ ID NO: 2 or a position corresponding thereto.

Examples of the mutant of the present invention include mutants in which any of the following amino acid residues (i) to (v) is substituted in the amino acid sequence of SEQ ID NO: 2 or an amino acid sequence having at least 90% identity thereto:
(i) an amino acid residue at position 295 or a position corresponding thereto and an amino acid residue at position 303 or a position corresponding thereto;
(ii) an amino acid residue at position 295 or a position corresponding thereto, an amino acid residue at position 296 or a position corresponding thereto, and an amino acid residue at position 303 or a position corresponding thereto;
(iii) an amino acid residue at position 331 or a position corresponding thereto;
(iv) an amino acid residue at position 295 or a position corresponding thereto, an amino acid residue at position 303 or a position corresponding thereto, and an amino acid residue at position 331 or a position corresponding thereto; and
(v) an amino acid residue at position 295 or a position corresponding thereto, an amino acid residue at position 296 or a position corresponding thereto, an amino acid residue at position 303 or a position corresponding thereto, and an amino acid residue at position 331 or a position corresponding thereto.

Preferred examples of such mutants include those in which tyrosine at position 295 or a position corresponding thereto is substituted with glutamine, glutamic acid, glycine, histidine, leucine, methionine, serine, threonine, or valine; and those in which tyrosine at position 303 or a position corresponding thereto is substituted with alanine, arginine, asparagine, aspartic acid, cysteine, glutamine, glutamic acid, glycine, histidine, isoleucine, leucine, lysine, methionine, serine, threonine, or valine; and more preferably those in which tyrosine at position 295 or a position corresponding thereto is substituted with glutamine, glutamic acid, glycine, histidine, leucine, methionine, serine, threonine, or valine; those in which alanine at position 296 or a position corresponding thereto is substituted with tyrosine; and those in which tyrosine at position 303 or a position corresponding thereto is substituted with alanine, arginine, asparagine, aspartic acid, cysteine, glutamine, glutamic acid, glycine, histidine, isoleucine, leucine, lysine, methionine, serine, threonine, or valine. Alternatively, preferred are those in which threonine at position 331 or a position corresponding thereto is substituted with serine; and more preferred are those in which tyrosine at position 295 or a position corresponding thereto is substituted with glutamine, glutamic acid, glycine, histidine, leucine, methionine, serine, threonine, or valine; those in which tyrosine at position 303 or a position corresponding thereto is substituted with alanine, arginine, asparagine, aspartic acid, cysteine, glutamine, glutamic acid, glycine, histidine, isoleucine, leucine, lysine, methionine, serine, threonine, or valine; and those in which threonine at position 331 or a position corresponding thereto is substituted with serine. Even more preferred are those in which tyrosine at position 295 or a position corresponding thereto is substituted with glutamine, glutamic acid, glycine, histidine, leucine, methionine, serine, threonine, or valine; those in which alanine at position 296 or a position corresponding thereto is substituted with tyrosine; those in which tyrosine at position 303 or a position corresponding thereto is substituted with alanine, arginine, asparagine, aspartic acid, cysteine, glutamine, glutamic acid, glycine, histidine, isoleucine, leucine, lysine, methionine, serine, threonine, or valine; and those in which threonine at position 331 or a position corresponding thereto is substituted with serine.

Therefore, in a preferred embodiment, the mutant of the present invention is an α-amylase mutant containing any amino acid substitution among Y295Q+Y303N, Y295E+Y303N, Y295G+Y303N, Y295L+Y303N, Y295M+Y303N, Y295S+Y303N, Y295T+Y303N, Y295V+Y303N, Y295H+A296Y+Y303A, Y295H+A296Y+Y303R, Y295H+A296Y+Y303N, Y295H+A296Y+Y303D, Y295H+A296Y+Y303C, Y295H+A296Y+Y303Q, Y295H+A296Y+Y303E, Y295H+A296Y+Y303G, Y295H+A296Y+Y303H, +Y295H+A296Y+Y303I, Y295H+A296Y+Y303L, Y295H+A296Y+Y303K, Y295H+A296Y+Y303M, Y295H+A296Y+Y303S, Y295H+A296Y+Y303T, Y295H+A296Y+Y303V, T331S, Y295E+Y303N+T331S, and Y295H+A296Y+Y303N+T331S, in an amino acid sequence having at least 90% identity to the amino acid sequence of SEQ ID NO: 2. More preferred among these is an α-amylase mutant containing any amino acid substitution among Y295H+A296Y+Y303A, Y295H+A296Y+Y303R, Y295H+A296Y+Y303N, Y295H+A296Y+Y303D, Y295H+A296Y+Y303C, Y295H+A296Y+Y303Q, Y295H+A296Y+Y303E, Y295H+A296Y+Y303G, Y295H+A296Y+Y303H, +Y295H+A296Y+Y303I, Y295H+A296Y+Y303L, Y295H+A296Y+Y303K, Y295H+A296Y+Y303M, Y295H+A296Y+Y303S, Y295H+A296Y+Y303T, Y295H+A296Y+Y303V, T331S, Y295E+Y303N+T331S, and Y295H+A296Y+Y303N+T331S, in an amino acid sequence having at least 90% identity to the amino acid sequence of SEQ ID NO: 2; and even more preferred is an α-amylase mutant containing the amino acid substitution Y295H+A296Y+Y303N+T331S in an amino acid sequence having at least 90% identity to the amino acid sequence of SEQ ID NO: 2.

### <Polynucleotide Encoding the Mutant of the Present Invention>

The mutant of the present invention can be produced by using various mutagenesis techniques known in this technical field. For example, the mutant of the present invention can be produced by mutating a polynucleotide encoding an amino acid residue to be substituted in a parent α-amylase gene encoding its reference amino acid sequence (reference α-amylase gene) to a polynucleotide encoding the substituted amino acid residue, and further allowing the mutated gene to express a mutant.

The polynucleotide encoding the mutant of the present invention can be in the form of single- or double-stranded DNA, RNA, or an artificial nucleic acid, or may be cDNA or chemically synthesized intron-free DNA.

In the present invention, as the means for mutating an amino acid residue of the parent α-amylase, various mutagenesis techniques known in this technical field can be used. For example, the polynucleotide encoding the mutant of the present invention can be obtained by mutating, in a polynucleotide encoding the amino acid residue of the parent α-amylase (hereinafter also referred to as the “parent gene”), a nucleotide sequence encoding an amino acid residue to be mutated to a nucleotide sequence encoding the mutated amino acid residue.

Introduction of the target mutation into the parent gene can be basically performed by various site-specific mutagenesis methods well-known to a person skilled in the art. The site-specific mutagenesis method can be performed by any method, such as an inverse PCR method or an annealing method. It is also possible to use commercially available site-specific mutagenesis kits (e.g., Stratagene's QuickChange II Site-Directed Mutagenesis Kit and QuickChange Multi Site-Directed Mutagenesis Kit, or the like).

Most commonly, site-specific mutagenesis of the parent gene can be performed by using a mutation primer containing the nucleotide mutation to be introduced. The mutation primer may be designed to be annealed to a region containing a nucleotide sequence encoding an amino acid residue to be mutated in the parent gene, and to contain a nucleotide sequence having a nucleotide sequence (codon) encoding the mutated amino acid residue in place of the nucleotide sequence (codon) encoding the amino acid residue to be mutated. The nucleotide sequences (codons) encoding the unmutated or mutated amino acid residues can be appropriately recognized and selected by a person skilled in the art based on ordinary textbooks and the like. Alternatively, site-specific mutagenesis can also be performed by using a method in which DNA fragments, obtained by amplifying the upstream and downstream sides of the mutation site separately using two complementary primers containing the nucleotide mutation to be introduced, are linked into one by SOE (splicing by overlap extension)-PCR (Gene, 1989, 77(1): pp. 61-68).

A template DNA containing the parent gene can be prepared by extracting genome DNA from a microorganism producing the α-amylase described above by a standard method, or extracting RNA and synthesizing cDNA with reverse transcription. Alternatively, a corresponding nucleotide sequence may be chemically synthesized based on the amino acid sequence of the parent α-amylase, and used as the template DNA. A DNA sequence containing a base sequence encoding BkoAmy, described above as an α-amylase, is shown in SEQ ID NO: 1.

A mutation primer can be produced by well-known oligonucleotide synthesis methods, such as the phosphoramidite method (Nucleic Acids Research, 1989, 17: 7059-7071). Such primer synthesis can also be performed using, for example, a commercially available oligonucleotide synthesizer (e.g., ABI). Using a primer set containing the mutation primer, site-specific mutagenesis as described above can be carried out using the parent gene as the template DNA, thereby obtaining a polynucleotide encoding the mutant of the present invention having the target mutation.

The polynucleotide encoding the mutant of the present invention can contain single- or double-stranded DNA, cDNA, RNA, or other artificial nucleic acids. The DNA, cDNA, and RNA may be chemically synthesized. The polynucleotide may contain nucleotide sequences of untranslated regions (UTRs) in addition to open reading frames (ORFs). The codon of the polynucleotide may be optimized depending on the species of the transformant used for the production of the mutant of the present invention. Information on codons used by various organisms is available from the Codon Usage Database ([www.kazusa.or.jp/codon/]).

### <Vector or DNA Fragment>

The obtained polynucleotide encoding the mutant of the present invention can be incorporated into a vector. The type of vector to contain the polynucleotide is not particularly limited, and any vector, such as a plasmid, phage, phagemid, cosmid, virus, YAC vector, or shuttle vector, may be used. The vector is not limited, but is preferably a vector which can be amplified in bacteria, preferably Bacillus bacteria (e.g., Bacillus subtilis or mutant strains thereof), and more preferably an expression vector which can induce the expression of transgenes in Bacillus bacteria. Among these, shuttle vectors, which are vectors replicable in Bacillus bacteria and any other organisms, can be preferably used in the recombinant production of the mutant of the present invention. Examples of preferred vectors include, but are not limited to, pHA3040SP64, pHSP64R, or pASP64 (JP-B-3492935), pHY300PLK (an expression vector which can transform both Escherichia coli and Bacillus subtilis; Jpn J Genet, 1985, 60: 235-243), pAC3 (Nucleic Acids Res, 1988, 16: 8732), and other shuttle vectors; pUB110 (J Bacteriol, 1978, 134: 318-329), pTA10607 (Plasmid, 1987, 18: 8-15), and other plasmid vectors which can be used in the transformation of Bacillus bacteria; and the like. Other usable examples include Escherichia coli-derived plasmid vectors (e.g., pET22b(+), pBR322, pBR325, pUC57, pUC118, pUC119, pUC18, pUC19, and pBluescript, and the like).

The above vector may contain a DNA replication initiation region or a DNA region containing a replication origin. Alternatively, in the above vector, a regulatory sequence, such as a promoter region for initiating the transcription of the gene, a terminator region, or a secretion signal region for secreting the expressed protein outside the cell, may be operably linked to the upstream of the polynucleotide encoding the mutant of the present invention (i.e., mutant gene). The phrase that a gene and a regulatory sequence are “operably linked” means that the gene and the regulatory region are arranged so that the gene can be expressed under the control of the regulatory region.

The type of regulatory sequence, such as a promoter region, a terminator, or a secretion signal region mentioned above, is not particularly limited, and generally used promoters and secretion signal sequences can be appropriately selected depending on the host for introduction. Examples of preferred regulatory sequences that can be incorporated into the vector include the promoter, secretion signal sequence, and the like of the cellulase gene of Bacillus sp. KSM-S237 strain.

Alternatively, a marker gene (e.g., a gene resistant to drugs, such as ampicillin, neomycin, kanamycin, and chloramphenicol) for selecting the host into which the vector of the present invention is appropriately introduced may be further incorporated into the vector. Alternatively, when an auxotroph is used as the host, a gene encoding the desired nutritional synthetic enzyme may be incorporated as a marker gene into the vector. Alternatively, when a selective culture medium in which a specific metabolism is required for growth, is used, a gene associated with the metabolism may be incorporated as a marker gene into the vector. Examples of such metabolism-related gene include acetamidase genes for utilizing acetamide as a nitrogen source.

The polynucleotide encoding the mutant of the present invention, a regulatory sequence, and a marker gene can be linked by a method known in the art, such as SOE (splicing by overlap extension)-PCR (Gene, 1989, 77: 61-68). Procedures for introducing the linked fragment into the vector are well known in the art.

### <Transformed Cell>

The transformed cell of the present invention can be obtained by introducing a vector containing the polynucleotide encoding the mutant of the present invention into a host, or by introducing a DNA fragment containing the polynucleotide encoding the mutant of the present invention into the genome of the host.

Examples of the host cells include microorganisms, such as bacteria and filamentous fungi. Examples of bacteria include Escherichia coli and bacteria belonging to the genera Staphylococcus, Enterococcus, Listeria, and Bacillus. Preferred among these are Escherichia coli and Bacillus bacteria (e.g., Bacillus subtilis Marburg No. 168 (Bacillus subtilis 168 strain) or mutant strains thereof). Examples of Bacillus subtilis mutant strains include the nine-protease-deficient strain KA8AX described in J. Biosci. Bioeng., 2007, 104(2): 135-143, and the eight-protease-deficient strain with improved protein folding efficiency, D8PA strain, described in Biotechnol. Lett., 2011, 33(9): 1847-1852. Examples of filamentous fungi include Trichoderma, Aspergillus, Rizhopus, and the like.

Methods commonly used in the art, such as the protoplast method and the electroporation method, can be used to introduce the vector into the host. Strains with appropriate introduction are selected using marker gene expression, auxotrophy, and the like as indices, whereby the target transformant into which the vector is introduced can be obtained.

Alternatively, a fragment obtained by linking the polynucleotide encoding the mutant of the present invention, a regulatory sequence, and a marker gene can also be introduced directly into the genome of the host. For example, a DNA fragment in which sequences complementary to the genome of the host are added to both ends of the linked fragment is constructed by the SOE-PCR method or the like, and this DNA fragment is then introduced into the host to induce homologous recombination between the host genome and the DNA fragment, whereby the polynucleotide encoding the mutant of the present invention is introduced into the genome of the host.

When the thus-obtained transformant into which the polynucleotide encoding the mutant of the present invention, or a vector containing the polynucleotide is introduced, is cultured in a suitable culture medium, the gene encoding the protein on the vector is expressed to produce the mutant of the present invention. The culture medium used for culturing the transformant can be appropriately selected by a person skilled in the art depending on the type of microorganism of the transformant.

Alternatively, the mutant of the present invention may be expressed from the polynucleotide encoding the mutant of the present invention or a transcript thereof using a cell-free translation system. The “cell-free translation system” is such that reagents, such as amino acids, necessary for the protein translation are added to a suspension obtained by mechanically destroying a cell, which serves as the host, to construct an in vitro transcription-translation system or an in vitro translation system.

The mutant of the present invention produced in the above culture or cell-free translation system can be isolated or purified by using general methods used for protein purification, such as centrifugation, ammonium sulfate precipitation, gel chromatography, ion-exchange chromatography, and affinity chromatography, singly or in a suitable combination. In this case, when the gene encoding the α-amylase mutant of the present invention is operably linked to a secretion signal sequence on the vector in the transformant, the produced protein is secreted extracellularly, and can be thus more easily collected from the culture. The protein collected from the culture may be further purified by known means.

The thus-obtained mutant of the present invention has enhanced amylolytic activity at low temperatures in comparison with the parent α-amylase.

The “amylolytic activity” can be determined by measuring the amount of reducing ends produced by the enzymatic degradation of starch. The determination method is not limited thereto; for example, the amylolytic activity can also be determined by measuring the release of dye by the enzymatic degradation of dye-crosslinked starch, such as Phadebas. There is a correlation between the amylolytic activity measured using Phadebas and the cleaning performance when used as a cleaning agent.

The mutant of the present invention is useful as an enzyme to be contained in various cleaning compositions, and particularly useful as an enzyme to be contained in cleaning compositions suitable for low-temperature cleaning.

Examples of the “low temperature” as mentioned herein include 40° C. or lower, 35° C. or lower, 30° C. or lower, and 25° C. or lower, and also include 5° C. or higher, 10° C. or higher, and 15° C. or higher. Other examples include from 5 to 40° C., from 10 to 35° C., from 15 to 30° C., and from 15 to 25° C.

The amount of the mutant of the present invention contained in the cleaning composition is not particularly limited as long as the protein can exhibit activity. For example, the amount of the mutant per kg of the cleaning composition is preferably 1 mg or more, more preferably 10 mg or more, and even more preferably 50 mg or more, as well as preferably 5,000 mg or less, more preferably 1,000 mg or less, and even more preferably 500 mg or less. The amount of the mutant is also preferably from 1 to 5,000 mg, more preferably from 10 to 1,000 mg, and even more preferably from 50 to 500 mg.

In the cleaning composition, various enzymes other than the mutant of the present invention can be used in combination. Examples include hydrolases, oxidases, reductases, transferases, lyases, isomerases, ligases, synthetases, and the like. Preferred among these are amylases which are different from the protein of the present invention, proteases, cellulases, keratinases, esterases, cutinases, lipases, pullulanases, pectinases, mannanases, glucosidases, glucanases, cholesterol oxidases, peroxidases, laccases, and the like; and particularly preferred are proteases, cellulases, amylases, and lipases.

Examples of proteases include commercially available Alcalase, Esperase, Everlase, Savinase, Kannase, and Progress Uno (registered trademarks; Novozymes A/S), PREFERENZ, EFFECTENZ, and EXCELLENZ (registered trademarks; DuPont), Lavergy (registered trademark; BASF), KAP (Kao Corporation), and the like.

Examples of cellulases include Celluclean and Carezyme (registered trademarks; Novozymes A/S); KAC, the alkaline cellulase produced by Bacillus sp. KSM-S237 strain described in JP-A-10-313859, and the mutant alkaline cellulase described in JP-A-2003-313592 (Kao Corporation); and the like.

Examples of amylases include Termamyl, Duramyl, Stainzyme, Stainzyme Plus, and Amplify Prime (registered trademarks; Novozymes A/S), PREFERENZ and EFFECTENZ (registered trademarks; DuPont), KAM (Kao Corporation), and the like.

Examples of lipases include Lipolase and Lipex (registered trademarks; Novozymes A/S), and the like.

Known cleaning agent components can be contained in the cleaning composition, and examples of such known cleaning agent components include the following.

(1) Surfactant

A surfactant may be contained in an amount of from 0.5 to 60 mass % in the cleaning composition, and preferably from 10 to 45 mass % particularly in a powder cleaning composition, and from 20 to 90 mass % in a liquid cleaning composition. When the cleaning composition of the present invention is a clothing cleaning agent for laundry or a cleaning agent for an automatic dishwasher, the surfactant is generally contained in an amount of from 1 to 10 mass %, and preferably from 1 to 5 mass %.

Examples of the surfactant used in the cleaning composition include one or a combination of anionic surfactants, nonionic surfactants, amphoteric surfactants, and cationic surfactants; and anionic surfactants and nonionic surfactants are preferred.

Examples of preferred anionic surfactants include sulfate ester salts of alcohols having from 10 to 18 carbon atoms, sulfate ester salts of alkoxylated alcohols having from 8 to 20 carbon atoms, alkylbenzene sulfonate, paraffin sulfonate, α-olefin sulfonate, internal olefin sulfonate, α-sulfo fatty acid salts, α-sulfo fatty acid alkyl ester salts, and fatty acid salts. In the present invention, particularly preferred is one or more anionic surfactants selected from the group consisting of linear alkylbenzene sulfonate with an alkyl chain having from 10 to 14 carbon atoms, more preferably from 12 to 14 carbon atoms, and internal olefin sulfonate with an alkylene chain having from 12 to 20 carbon atoms, more preferably from 16 to 18 carbon atoms. Alkali metal salts and amines are preferable as counterions, and sodium and/or potassium, monoethanolamine, and diethanolamine are particularly preferred. For internal olefin sulfonic acid, reference can be made to, for example, WO 2017/098637.

Preferred nonionic surfactants are polyoxyalkylene alkyl (from 8 to 20 carbon atoms) ether, alkyl polyglycoside, polyoxyalkylene alkyl (from 8 to 20 carbon atoms) phenyl ether, polyoxyalkylene sorbitan fatty acid (from 8 to 22 carbon atoms) ester, polyoxyalkylene glycol fatty acid (from 8 to 22 carbon atoms) ester, and polyoxyethylene polyoxypropylene block polymers. In particular, preferred nonionic surfactants are polyoxyalkylene alkyl ethers obtained by adding 4 to 20 moles of alkylene oxides, such as ethylene oxide and propylene oxide, to alcohols having from 10 to 18 carbon atoms [an HLB value (calculated by the Griffin method) of from 10.5 to 15.0, and preferably from 11.0 to 14.5].

(2) Divalent Metal Ion Scavenger

A divalent metal ion scavenger may be contained in an amount of from 0.01 to 50 mass %, and preferably from 5 to 40 mass %. Examples of the divalent metal ion scavenger used in the cleaning composition of the present invention include condensed phosphates, such as tripolyphosphates, pyrophosphates, and orthophosphates; aluminosilicates, such as zeolites; synthetic layered crystalline silicates, nitrilotriacetates, ethylenediaminetetraacetates, citrates, isocitrates, polyacetal carboxylates, and the like. Among these, crystalline aluminosilicates (synthetic zeolites) are particularly preferred. Among A-, X-, and P-type zeolites, A-type zeolites are particularly preferred. As synthetic zeolites, those having an average primary particle size of from 0.1 to 10 μm, particularly from 0.1 to 5 μm, are preferably used.

(3) Alkali Agent

An alkali agent may be contained in an amount of from 0.01 to 80 mass %, preferably from 1 to 40 mass %. In the case of powder cleaning agents, examples of alkali agents include alkali metal carbonates, such as sodium carbonate, collectively called dense ash or light ash; and amorphous alkali metal silicates, such as JIS Nos. 1, 2, and 3. These inorganic alkali agents are effective in the formation of the particle skeleton during drying of cleaning agent, and relatively hard cleaning agents having excellent flowability can be obtained. Examples of other alkalis include sodium sesquicarbonate, sodium hydrogencarbonate, and the like. In addition, phosphates, such as tripolyphosphates, also have the action as alkali agents. As alkali agents used in liquid cleaning agents, sodium hydroxide and mono-, di-, or triethanolamine can be used, in addition to the alkali agents mentioned above, and they can also be used as counterions of activators.

(4) Anti-Redeposition Agent

An anti-redeposition agent may be contained in an amount of from 0.001 to 10 mass %, preferably from 1 to 5 mass %. Examples of the anti-redeposition agent used in the cleaning composition of the present invention include polyethylene glycol, carboxylic acid-based polymers, polyvinyl alcohol, polyvinylpyrrolidone, and the like. Among these, carboxylic acid-based polymers have the function of scavenging metal ions and the ability to disperse solid particle stains from the clothing into the laundry bath, as well as the anti-redeposition ability. Carboxylic acid-based polymers are homopolymers or copolymers of acrylic acid, methacrylic acid, itaconic acid, or the like. Preferred copolymers are copolymers of the above monomers and maleic acid, and those with a molecular weight of from several thousands to a hundred thousand are preferred. In addition to the carboxylic acid-based polymers mentioned above, polymers such as polyglycidylates, cellulose derivatives such as carboxymethyl cellulose, and amino carboxylic acid-based polymers such as polyaspartic acid are also preferred because they have metal ion scavenging, dispersion, and anti-redeposition ability.

(5) Bleaching Agent

A bleaching agent, such as hydrogen peroxide or percarbonate, may be preferably contained in an amount of from 1 to 10 mass %. When the bleaching agent is used, tetraacetylethylenediamine (TAED) or the bleach activator described in JP-A-6-316700 can be contained in an amount of from 0.01 to 10 mass %.

(6) Fluorescent Agent

Examples of the fluorescent agent used in the cleaning composition include biphenyl-type fluorescent agents (e.g., Tinopal CBS-X and the like) and stilbene-type fluorescent agents (e.g., a DM-type fluorescent dye and the like). The fluorescent agent is preferably contained in an amount of from 0.001 to 2 mass %.

(7) Other Components

The cleaning composition may contain builders, softeners, reducing agents (e.g., sulfite), foam inhibitors (e.g., silicone), fragrances, antibacterial and antifungal agents (e.g., Proxel [trade name] and benzoic acid), and other additives known in the field of clothing cleaning agents.

The cleaning composition can be produced in accordance with a standard method by combining the protein of the present invention obtained by the above method and the known cleaning components mentioned above. The form of the cleaning agent can be selected depending on the application. For example, the cleaning agent can be in the form of liquid, powder, granules, paste, solids, or the like.

The thus-obtained cleaning composition can be used as a clothing cleaning agent, a dishwashing cleaning agent, a bleaching agent, a cleaning agent for hard surface cleaning, a drain cleaning agent, a denture cleaning agent, a disinfecting cleaning agent for medical instruments, or the like; preferably a clothing cleaning agent and a dishwashing cleaning agent; and more preferably a clothing cleaning agent for laundry (laundry cleaning agent), a dishwashing cleaning agent for hand washing, and a cleaning agent for an automatic dishwasher.

The cleaning composition is suitable for use at 40° C. or lower, 35° C. or lower, 30° C. or lower, or 25° C. or lower, and at 5° C. or higher, 10° C. or higher, or 15° C. or higher. The cleaning composition is also suitable for use at from 5 to 40° C., from 10 to 35° C., from 15 to 30° C., or from 15 to 25° C. Preferred use modes include use for low-temperature (from 15 to 30° C.) cleaning in laundries, and low-temperature (from 15 to 30° C.) cleaning by the automatic dishwasher.

Regarding the embodiments described above, the present invention further discloses the following aspects.

<1> An α-amylase mutant in which an amino acid residue at a position of the following (a) and (b); (c); or (a), (b), and (c), in the amino acid sequence of SEQ ID NO: 2 or an amino acid sequence having at least 90% identity thereto is substituted with another amino acid residue:
(a) position 303 of the amino acid sequence of SEQ ID NO: 2 or a position corresponding thereto;
(b) one or more positions selected from the group consisting of position 295 and position 296 of the amino acid sequence of SEQ ID NO: 2 and positions corresponding thereto; or
(c) position 331 of the amino acid sequence of SEQ ID NO: 2 or a position corresponding thereto.

<2> The mutant according to <1>, wherein the substitution of the amino acid residue at the position of (a) is substitution to A, R, N, D, C, Q, E, G, H, I, L, K, M, S, T, or V; the substitution of the amino acid residue at the position of (b) is one or both of (b-1) substitution of an amino acid residue at position 295 or a position corresponding thereto to Q, E, G, H, L, M, S, T, or V, and (b-2) substitution of an amino acid residue at position 296 or a position corresponding thereto to Y; and the substitution of the amino acid residue at the position of (c) is substitution to S.

<3> The mutant according to <1> or <2>, wherein the substitution of the amino acid residue is substitution of any of the following amino acid residues (i) to (v):
(i) an amino acid residue at position 295 or a position corresponding thereto and an amino acid residue at position 303 or a position corresponding thereto;
(ii) an amino acid residue at position 295 or a position corresponding thereto, an amino acid residue at position 296 or a position corresponding thereto, and an amino acid residue at position 303 or a position corresponding thereto;
(iii) an amino acid residue at position 331 or a position corresponding thereto;
(iv) an amino acid residue at position 295 or a position corresponding thereto, an amino acid residue at position 303 or a position corresponding thereto, and an amino acid residue at position 331 or a position corresponding thereto; and
(v) an amino acid residue at position 295 or a position corresponding thereto, an amino acid residue at position 296 or a position corresponding thereto, an amino acid residue at position 303 or a position corresponding thereto, and an amino acid residue at position 331 or a position corresponding thereto.

<4> The mutant according to <3>, wherein the substitution of the amino acid residue is substitution of the amino acid residue of (i) above.

<5> The mutant according to <3>, wherein the substitution of the amino acid residue is substitution of the amino acid residue of (ii) above.

<6> The mutant according to <3>, wherein the substitution of the amino acid residue is substitution of the amino acid residue of (iii) above.

<7> The mutant according to <3>, wherein the substitution of the amino acid residue is substitution of the amino acid residue of (iv) above.

<8> The mutant according to <3>, wherein the substitution of the amino acid residue is substitution of the amino acid residue of (v) above.

<9> The mutant according to any one of <1> to <3>, wherein the mutant contains any amino acid substitution among Y295Q+Y303N, Y295E+Y303N, Y295G+Y303N, Y295L+Y303N, Y295M+Y303N, Y295S+Y303N, Y295T+Y303N, Y295V+Y303N, Y295H+A296Y+Y303A, Y295H+A296Y+Y303R, Y295H+A296Y+Y303N, Y295H+A296Y+Y303D, Y295H+A296Y+Y303C, Y295H+A296Y+Y303Q, Y295H+A296Y+Y303E, Y295H+A296Y+Y303G, Y295H+A296Y+Y303H, +Y295H+A296Y+Y303I, Y295H+A296Y+Y303L, Y295H+A296Y+Y303K, Y295H+A296Y+Y303M, Y295H+A296Y+Y303S, Y295H+A296Y+Y303T, Y295H+A296Y+Y303V, T331S, Y295E+Y303N+T331S, and Y295H+A296Y+Y303N+T331S.

<10> The mutant according to any one of <1> to <9>, wherein amylolytic activity, preferably amylolytic activity at from 5 to 40° C., is enhanced in comparison with a parent α-amylase.

<11> A method for producing an α-amylase mutant, comprising substituting, with another amino acid residue, an amino acid residue at a position of the following (a) and (b); (c); or (a), (b), and (c), in the amino acid sequence of SEQ ID NO: 2 or an amino acid sequence having at least 90% identity thereto:
(a) position 303 of the amino acid sequence of SEQ ID NO: 2 or a position corresponding thereto;
(b) one or more positions selected from the group consisting of position 295 and position 296 of the amino acid sequence of SEQ ID NO: 2 and positions corresponding thereto; or
(c) position 331 of the amino acid sequence of SEQ ID NO: 2 or a position corresponding thereto.

<12> The method according to <11>, wherein the substitution of the amino acid residue at the position of (a) is substitution to A, R, N, D, C, Q, E, G, H, I, L, K, M, S, T, or V; the substitution of the amino acid residue at the position of (b) is one or both of (b-1) substitution of an amino acid residue at position 295 or a position corresponding thereto to Q, E, G, H, L, M, S, T, or V, and (b-2) substitution of an amino acid residue at position 296 or a position corresponding thereto to Y; and the substitution of the amino acid residue at the position of (c) is substitution to S.

<13> A method for enhancing amylolytic activity, comprising substituting, with another amino acid residue, an amino acid residue at a position of the following (a) and (b); (c); or (a), (b), and (c), in the amino acid sequence of SEQ ID NO: 2 or an amino acid sequence having at least 90% identity thereto:
(a) position 303 of the amino acid sequence of SEQ ID NO: 2 or a position corresponding thereto;
(b) one or more positions selected from the group consisting of position 295 and position 296 of the amino acid sequence of SEQ ID NO: 2 and positions corresponding thereto; or
(c) position 331 of the amino acid sequence of SEQ ID NO: 2 or a position corresponding thereto.

<14> The method according to <13>, wherein the substitution of the amino acid residue at the position of (a) is substitution to A, R, N, D, C, Q, E, G, H, I, L, K, M, S, T, or V; the substitution of the amino acid residue at the position of (b) is one or both of (b-1) substitution of an amino acid residue at position 295 or a position corresponding thereto to Q, E, G, H, L, M, S, T, or V, and (b-2) substitution of an amino acid residue at position 296 or a position corresponding thereto to Y; and the substitution of the amino acid residue at the position of (c) is substitution to S.

<15> A polynucleotide encoding the mutant according to any one of <1> to <10>.

<16> A vector or DNA fragment comprising the polynucleotide according to <15>.

<17> A transformed cell comprising the vector or DNA fragment according to <16>.

<18> The transformed cell according to <17>, which is a microorganism.

<19> The transformed cell according to <17> or <18>, which is Escherichia coli or Bacillus bacteria.

<20> A cleaning composition comprising the mutant according to any one of <1> to <10>.

<21> The cleaning composition according to <20>, which is a clothing cleaning agent or a dishwashing cleaning agent.

<22> The cleaning composition according to <21>, which is a clothing cleaning agent for laundry or a dishwashing cleaning agent for hand washing or an automatic dishwasher.

<23> The cleaning composition according to <21> or 22>, which is a powder or a liquid.

<24> The cleaning composition according to any one of <21> to <23>, which is used at a low temperature.

<25> The cleaning composition according to <24>, which is used at 40° C. or lower, 35° C. or lower, 30° C. or lower, or 25° C. or lower, and 5° C. or higher, 10° C. or higher, or 15° C. or higher, or used at from 5 to 40° C., from 10 to 35° C., from 15 to 30° C., or from 15 to 25° C.

<26> The cleaning composition according to <21>, which is used in low-temperature (from 15 to 30° C.) cleaning in laundries or low-temperature (from 15 to 30° C.) cleaning by the automatic dishwasher.

### Examples

The present invention is described in more detail below based on Examples; however, the present invention is not limited thereto.

(1) Construction of Amylase Expression Plasmid

PCR was performed using the BkoAmy gene (SEQ ID NO: 1) obtained by artificial gene synthesis as a template, a primer pair BKoAmy_fw/BkoAmy_rv (SEQ ID NOs: 11 and 12) and PrimeSTAR Max Premix (Takara Bio Inc.). Using the plasmid pHY-5237 described in Example 7 of NO 2006/068148 A1 as a template, PCR was similarly performed using a primer pair S237t_fw/S237s_rv (SEQ ID NOs: 13 and 14). Using each of the PCR products, the In-Fusion reaction was performed in accordance with the protocol of the In-Fusion, HD Cloning kit (Clontech). Using the In-Fusion reaction solution, Bacillus subtilis was transformed to construct plasmid pHY-BKoAmy (wild-type BKoAmy expression plasmid). Similarly, for AP1378, AA560, SP722, and Cspamy2, genes obtained by artificial gene synthesis (SEQ ID NOs: 3, 5, 7, and 9) were each used as a template, and PCR and In-Fusion reaction were performed using a primer pair AP1378_fw/AP1378_rv (SEQ ID NOs: 15 and 16), AA560_fw/AA560_rv (SEQ ID NOs: 17 and 18), SP722_fw/SP722_rv (SEQ ID NOs: 19 and 20), or Cspamy2_fw/Cspamy2_rv (SEQ ID NOs: 21 and 22). Using the In-Fusion reaction solution to transform Bacillus subtilis, plasmids pHY-AP1378, pHY-AA560, pHY-SP722, and pHY-Cspamy2 were constructed.

The mutant construction method was carried out by the following procedure. A forward primer having 15 bases of a sequence complementary to a reverse primer at the 5′-terminal and containing a mutant sequence, and the reverse primer having a base just before the mutant sequence at the 5′-terminal were used as a mutagenesis primer pair. The above plasmid pHY-BKoAmy or the BkoAmy mutant expression plasmid produced in this example was used as a template, and PCR was performed by using the mutagenesis primer pair. Using the PCR product, Bacillus subtilis was transformed to obtain a transformant retaining the target BkoAmy mutant expression plasmid.

(2) Transformation

The host used was Bacillus subtilis 168 strain (Bacillus subtilis Marburg No. 168 strain: Nature, 390, 1997, p. 249). The Bacillus subtilis 168 strain was inoculated in 1 mL of LB culture medium and cultured by shaking at 30° C. and 200 rpm overnight. 10 μL of the culture was inoculated in 1 mL of fresh LB culture medium and cultured at 37° C. and 200 rpm for 3 hours. The culture was centrifuged, and pellets were collected. 500 μL of SMMP (0.5 M sucrose, 20 mM disodium malate, 20 mM magnesium chloride hexahydrate, and 35% (w/v) antibiotic medium 3 (Difco)) containing 4 mg/mL lysozyme (SIGMA) was added to the pellets, followed by incubation at 37° C. for 1 hour. Next, the pellets were collected by centrifugation and suspended in 400 μL of SMMP. 33 μL of the suspension was mixed with DNA, and 100 μL of 40% PEG was further added, followed by stirring. Further, 350 μL of SMMP was added, followed by shaking at 30° C. for 1 hour. 200 μL of the resulting liquid was smeared to DM3 regeneration agar culture medium (0.8% agar (FUJIFILM Wako Pure Chemical Corporation), 0.5% disodium succinate hexahydrate, 0.5% casamino acids technical (Difco), 0.5% yeast extract, 0.35% monopotassium phosphate, 0.15% dipotassium phosphate, 0.5% glucose, 0.4% magnesium chloride hexahydrate, 0.01° bovine serum albumin (SIGMA), 0.5% carboxymethyl cellulose, 0.005% trypan blue (Merck), and an amino acid mixed solution (tryptophan, lysine, and methionine, each 10 μg/mL); % denotes (w/v) %) containing tetracycline (15 μg/mL, SIGMA), followed by incubation at 30° C. for 3 days, and the formed colonies were obtained.

(3) Enzyme Production Culture

The recombinant Bacillus subtilis colonies obtained in (2) were inoculated in a 96-deep-well plate into which 300 μL of LB culture medium supplemented with 15 ppm tetracycline was dispensed, and then cultured at 30° C. at 210 rpm overnight. Next day, 6 μL of the culture was inoculated in a 96-deep-well plate into which 100 μL of 2×L-maltose culture medium (2% tryptone, 1% yeast extract, 1% NaCl, 7.5% maltose, 7.5 ppm manganese sulfate pentahydrate, 0.04% calcium chloride dihydrate, and 15 ppm tetracycline; % denotes (w/v) %) was dispensed, and cultured at 30° C. at 210 rpm for 2 days. Then, the culture supernatant containing the enzyme produced from the bacterial cell was collected by centrifugation.

(4) Measurement of Protein Concentration of Culture Supernatant

For the measurement of the protein concentration of the culture supernatant, Protein Assay Rapid Kit Wako II (FUJIFILM Wako Pure Chemical Corporation) was used. The protein concentration of a culture supernatant of a strain introduced with pHY300PLK (Takara Bio Inc.) having no amylase expression cassette was used as a blank to calculate the amylase concentration of the culture supernatant.

(5) Amylolytic Activity Measurement

The amylolytic activity of each culture supernatant was measured using Phadebas (Phadebas AB). Phadebas is a tablet made of insoluble starch covalently bonded to a blue dye. The water-soluble blue dye is released in association with amylolysis by α-amylase. The concentration of the blue dye measured by the absorbance at 620 nm is proportional to the amylase activity in the sample.

One substrate tablet was suspended per 5 mL of 1/15 M phosphate buffer (pH: 7.4). 500 μL of the substrate suspension was dispensed into a 96-deep-well plate. An enzyme solution suitably diluted with 1/15 M phosphate buffer (pH: 7.4) was added, followed by mixing. After standing still at 20° C. for 30 minutes, 250 μL of a 10% aqueous citric acid solution was added to terminate the reaction. After centrifugation at 3,000 rpm for 20 minutes, 100 μL of the supernatant was transferred to a new 96-well plate, and the absorbance at 620 nm was measured. It was confirmed that each measured value was within the linear range of activity measurement. By subtracting the value of the blank (enzyme-free), amylolytic activity ΔA620 was calculated, and the resulting value was further divided by the added amylase concentration to determine specific activity ΔA620/ppm. The resulting value was further divided by the specific activity of wild-type BkoAmy to determine relative amylolytic activity.

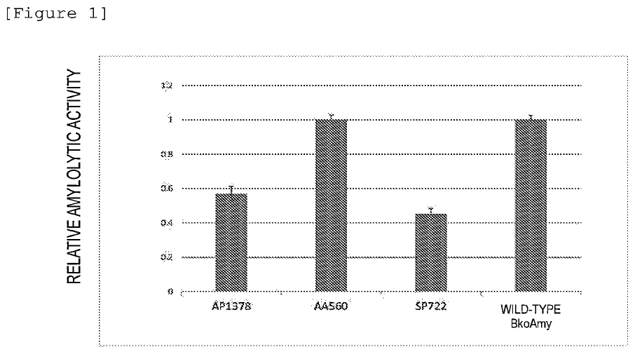

As a result of comparing amylolytic activity between wild-type BkoAmy and parent enzymes (AP1378, AA560, and SP722) of existing amylases for a cleansing agent derived from Bacillus bacteria, wild-type BkoAmy showed better amylolytic activity at 20° C. (FIG. 1).

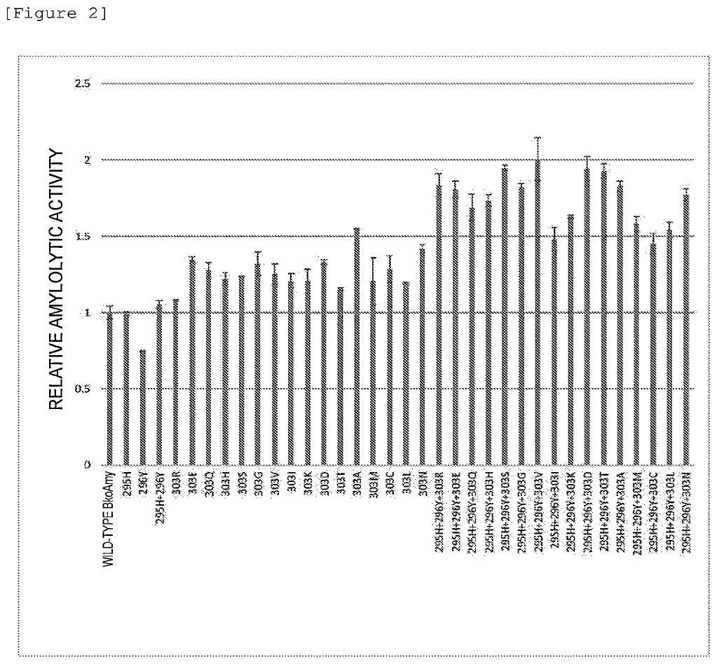

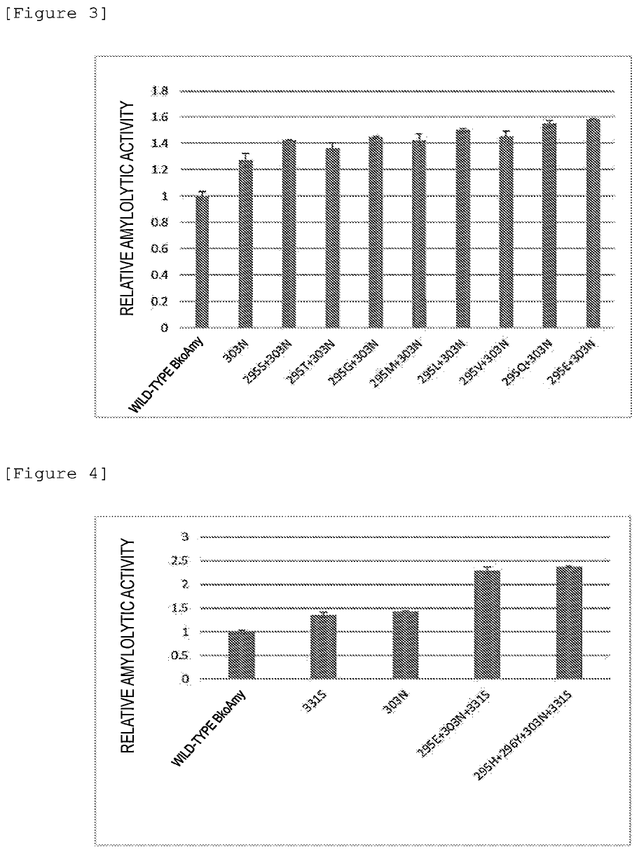

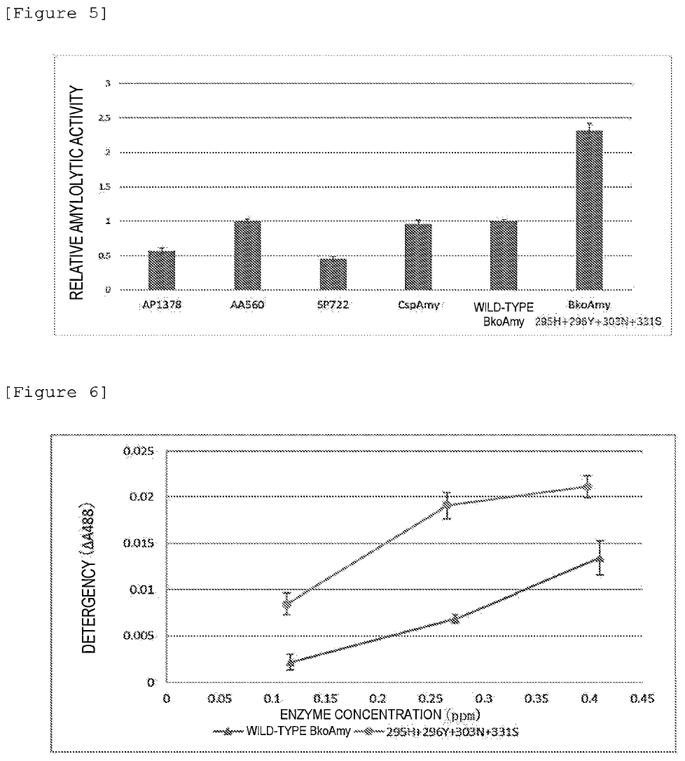

As a result of introducing a single mutation of 303R, E, Q, H, S, G, V, I, K, D, T, A, M, C, L, N, and 331S into wild-type BkoAmy, the amylolytic activity at 20° C. was enhanced (FIGS. 2 and 4). The activity was not enhanced by the mutation at position 295 and/or position 296, whereas unexpectedly, the amylolytic activity was significantly enhanced by combining 295H and 296Y with the above mutation at position 303 (FIG. 2). Further, the amylolytic activity was also enhanced by the combination of 303N with 295S, T, G, M, L, V, Q, or E (FIG. 3). In addition, as a result of introducing 331S mutation into mutants which were confirmed to have enhanced activity due to mutations at position 295 and/or position 296, and at position 303, even higher amylolytic activity was exhibited (FIG. 4). As a result of comparing the amylolytic activity of 295H+296Y+303N+331S mutant, which showed the highest amylolytic activity at this time, with the activity of the parent enzymes (AP1378, AA560, SP722, and Cspamy2) of the existing amylases for a cleaning agent, it was indicated that the above mutant had significantly higher activity (FIG. 5).

(6) Detergency Evaluation

A CS-26 stained cloth cut into a circular shape with a diameter of 5.5 mm was obtained from CFT. Two CS-26 circular stained cloths were inserted into each well of a 96-well assay plate, and 200 μL of Attack Antibacterial EX Super Clear Gel (Kao Corporation) diluted 1,200-fold with tap water was added to each well. 10 μL of a suitably diluted enzyme solution was added to each well, and the plate was sealed and shaken at 20° C. using a Cute Mixer at 1,200 rpm for 15 minutes. After the completion of cleaning, 100 μL of the cleaning liquid was transferred to a new 96-well assay plate, and the absorbance at 488 nm was measured. A blank was prepared by adding tap water in place of the enzyme solution, and the difference from the blank AA488 was determined as detergency.

295H+296Y+303N+331S, whose activity was largely enhanced as a result of mutagenesis, showed significantly higher detergency at low temperatures in comparison with wild-type Bkoamy (FIG. 6).

1. An α-amylase mutant in which an amino acid residue at a position of the following (a) and (b); (c); or (a), (b), and (c), in the amino acid sequence of SEQ ID NO: 2 or an amino acid sequence having at least 90% identity thereto is substituted with another amino acid residue:
(a) position 303 of the amino acid sequence of SEQ ID NO: 2 or a position corresponding thereto;
(b) one or more positions selected from the group consisting of position 295 and position 296 of the amino acid sequence of SEQ ID NO: 2 and positions corresponding thereto; or
(c) position 331 of the amino acid sequence of SEQ ID NO: 2 or a position corresponding thereto.

2. The mutant according to claim 1, wherein the substitution of the amino acid residue at the position of (a) is substitution to A, R, N, D, C, Q, E, G, H, I, L, K, M, S, T, or V; the substitution of the amino acid residue at the position of (b) is one or both of (b-1) substitution of an amino acid residue at position 295 or a position corresponding thereto to Q, E, G, H, L, M, S, T, or V, and (b-2) substitution of an amino acid residue at position 296 or a position corresponding thereto to Y; and the substitution of the amino acid residue at the position of (c) is substitution to S.

3. The mutant according to claim 1, wherein the substitution of the amino acid residue is substitution of any of the following amino acid residues (i) to (v):
(i) an amino acid residue at position 295 or a position corresponding thereto and an amino acid residue at position 303 or a position corresponding thereto;
(ii) an amino acid residue at position 295 or a position corresponding thereto, an amino acid residue at position 296 or a position corresponding thereto, and an amino acid residue at position 303 or a position corresponding thereto;
(iii) an amino acid residue at position 331 or a position corresponding thereto;
(iv) an amino acid residue at position 295 or a position corresponding thereto, an amino acid residue at position 303 or a position corresponding thereto, and an amino acid residue at position 331 or a position corresponding thereto; and
(v) an amino acid residue at position 295 or a position corresponding thereto, an amino acid residue at position 296 or a position corresponding thereto, an amino acid residue at position 303 or a position corresponding thereto, and an amino acid residue at position 331 or a position corresponding thereto.

4. A polynucleotide encoding the mutant according to claim 1.

5. A vector or DNA fragment comprising the polynucleotide according to claim 4.

6. A transformed cell comprising the vector or DNA fragment according to claim 5.

7. The transformed cell according to claim 6, which is a microorganism.

8. A cleaning composition comprising the mutant according to claim 1.

9. The cleaning composition according to claim 8, which is a clothing cleaning agent or a dishwashing cleaning agent.

10. The cleaning composition according to claim 9, which is used at a low temperature.

11. The cleaning composition according to claim 10, which is used at a temperature of from 5 to 40° C.

12. A method for producing an α-amylase mutant, comprising substituting, with another amino acid residue, an amino acid residue at a position of the following (a) and (b); (c); or (a), (b), and (c), in the amino acid sequence of SEQ ID NO: 2 or an amino acid sequence having at least 90% identity thereto:
(a) position 303 of the amino acid sequence of SEQ ID NO: 2 or a position corresponding thereto;
(b) one or more positions selected from the group consisting of position 295 and position 296 of the amino acid sequence of SEQ ID NO: 2 and positions corresponding thereto; or
(c) position 331 of the amino acid sequence of SEQ ID NO: 2 or a position corresponding thereto.

13. A method for enhancing amylolytic activity, comprising substituting, with another amino acid residue, an amino acid residue at a position of the following (a) and (b); (c); or (a), (b), and (c), in the amino acid sequence of SEQ ID NO: 2 or an amino acid sequence having at least 90% identity thereto:
(a) position 303 of the amino acid sequence of SEQ ID NO: 2 or a position corresponding thereto;
(b) one or more positions selected from the group consisting of position 295 and position 296 of the amino acid sequence of SEQ ID NO: 2 and positions corresponding thereto; or
(c) position 331 of the amino acid sequence of SEQ ID NO: 2 or a position corresponding thereto.

14. The method according to claim 12, wherein the substitution of the amino acid residue at the position of (a) is substitution to A, R, N, D, C, Q, E, G, H, I, L, K, M, S, T, or V; the substitution of the amino acid residue at the position of (b) is one or both of (b-1) substitution of an amino acid residue at position 295 or a position corresponding thereto to Q, E, G, H, L, M, S, T, or V, and (b-2) substitution of an amino acid residue at position 296 or a position corresponding thereto to Y; and the substitution of the amino acid residue at the position of (c) is substitution to S.

15. The method according to claim 13, wherein the substitution of the amino acid residue at the position of (a) is substitution to A, R, N, D, C, Q, E, G, H, I, L, K, M, S, T, or V; the substitution of the amino acid residue at the position of (b) is one or both of (b-1) substitution of an amino acid residue at position 295 or a position corresponding thereto to Q, E, G, H, L, M, S, T, or V, and (b-2) substitution of an amino acid residue at position 296 or a position corresponding thereto to Y; and the substitution of the amino acid residue at the position of (c) is substitution to S.


SEQUENCE LISTING (22 sequences)

SEQ ID NO: 1 - DNA, 1458 bp, Bacillus koreensis
  CDS (1)..(1455): Codon-optimized oligonucleotide
gcgactacgaatggcacaatgatgcagtatttcgaatggtatgttccgaacgatggacagcaatggaatagactgcgaacagatgcaccgtatttatcatccgttggcataacagcagtctggacacctccggcttataaaggaacctcacaagctgacgtaggctatgggccttacgatctttacgatctcggtgaattcaatcagaaagggacagtacgtacaaaatacggtactaaggccgagttgaaatccgccgttaatacgttacatagcaacggaatccaggtctatggcgatgtggtcatgaatcataaagcaggagcagactatacagagaatgttacggcagttgaggtgaatccgagtaatcggaaccaagaaacatcaggcgaatacaacattcaagcatggacaggctttaactttccagggaggggcacaacgtactcgaacttcaaatggcaatggtttcactttgacggaaccgattgggatcaaagccgttctctgtccagaatctttaaatttcgcggaacgggaaaggcttgggattgggaggtatctagcgagaatgggaactatgattatctgatgtatgctgacattgactatgatcatcccgatgttgtgaacgaaatgaagaagtggggagtttggtatgcgaatgaagtgggcttagatggctatcgcttggatgcagtcaagcatatcaagttttcgtttctgaaggattgggtagataacgctagagcagccacaggtaaagagatgttcactgtaggtgaatactggcaaaatgaccttggcgctctcaataactatctggcgaaagtcaattacaatcagtcactgtttgatgcaccgcttcattacaacttctatgctgcgtctacaggaggcggctattacgatatgcggaacatcttaaataacaccttagtggcgagtaatccgactaaagccgtgacattggtcgaaaatcacgatacacagcctgggcagtctcttgaaagcacggtgcaaccgtggttcaagccgttagcgtatgcctttatccttacccggtctggaggctatccatcagtcttttatggcgatatgtatggtaccaaaggtactactacgcgcgaaataccagccctgaaaagcaaaattgaacccctgctcaaagcgagaaaagactacgcttatggcacacaacgtgactacatcgacaaccctgacgtgattgggtggacacgagaaggtgattccacgaaagcgaaatcgggacttgcgacggtaattactgacggacctggcggaagtaaaaggatgtatgttggaacgagcaatgccggcgaagtgtggtatgatttgacgggtaatcgcacagacaaaattaccattggatctgatgggtacgcaacctttccagttaacggtgggtcagtctcagtatgggtccagcagtaa

SEQ ID NO: 2 - protein, 485 aa, Bacillus koreensis
ATTNGTMMQYFEWYVPNDGQQWNRLRTDAPYLSSVGITAVWTPPAYKGTSQADVGYGPYDLYDLGEFNQKGTVRTKYGTKAELKSAVNTLHSNGIQVYGDVVMNHKAGADYTENVTAVEVNPSNRNQETSGEYNIQAWTGFNFPGRGTTYSNFKWQWFHFDGTDWDQSRSLSRIFKFRGTGKAWDWEVSSENGNYDYLMYADIDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGYYDMRNILNNTLVASNPTKAVTLVENHDTQPGQSLESTVQPWFKPLAYAFILTRSGGYPSVFYGDMYGTKGTTTREIPALKSKIEPLLKARKDYAYGTQRDYIDNPDVIGWTREGDSTKAKSGLATVITDGPGGSKRMYVGTSNAGEVWYDLTGNRTDKITIGSDGYATFPVNGGSVSVWVQQ

SEQ ID NO: 3 - DNA, 1458 bp, Bacillus sp. KSM-1378
  CDS (1)..(1455): Codon-optimized oligonucleotide
catcataatggcacaaacggaacaatgatgcagtattttgaatggcatttaccgaatgatggcaaccattggaatagactgcgcgatgatgcagcgaatcttaaaagcaaaggcattacagctgtttggatcccgccggcctggaaaggaacatctcaaaacgatgtgggctatggagcttacgatctgtacgatctgggcgaatttaatcagaaaggaacagtcagaacaaaatatggcacacgctctcaattacagggagccgtgacatcactgaaaaataacggcattcaagtctatggagatgttgtgatgaatcataaaggcggagcagatggcacagaaatggttaatgcggttgaagtgaacagaagcaatcgcaaccaggaaatttctggagaatatacaatcgaagcatggacaaaatttgattttccgggcagaggaaatacacattcaaactttaaatggcgctggtatcattttgatggcacagattgggatcaaagcagacaactgcagaacaaaatctacaaatttcgcggcacaggaaaagcttgggattgggaagtggatatcgaaaacggaaactacgattacctgatgtacgccgatattgatatggatcatccggaagtcatcaatgaactgagaaactggggcgtttggtacacaaacacacttaacctggatggatttcgcattgatgcggtcaaacatatcaaatactcatacacaagagattggctgacacatgttcgcaatacaacaggcaaaccgatgtttgcagtggcggaattttggaaaaacgatcttgctgccatcgaaaactacctgaacaaaacatcatggaatcatagcgtctttgatgttccgcttcattacaacctgtacaacgcttctaactcaggcggatattttgatatgagaaacatccttaacggctctgtcgttcagaaacatccgatccatgcagtgacatttgtcgataatcatgattcacaaccgggagaagcgctggaatcttttgtgcagtcatggtttaaaccgcttgcttatgccctgattcttacacgcgaacaaggctatccgagcgtgttttatggcgattattatggaattccgacacatggagtcccgagcatgaaatctaaaatcgatccgttactgcaagcaagacagacatatgcgtatggaacacagcatgattactttgatcatcatgatatcatcggctggacacgcgaaggagattcaagccatccgaattcaggccttgcaacaatcatgagcgatggaccgggcggaaacaaatggatgtatgttggcaaacataaagcgggacaagtgtggagagatattacaggcaatcgcagcggaacagtcacaatcaacgctgatggctggggaaattttacagttaacggcggagccgtctctgtttgggtgaaacagtaa

SEQ ID NO: 4 - protein, 485 aa, Bacillus sp. KSM-1378
HHNGTNGTMMQYFEWHLPNDGNHWNRLRDDAANLKSKGITAVWIPPAWKGTSQNDVGYGAYDLYDLGEFNQKGTVRTKYGTRSQLQGAVTSLKNNGIQVYGDVVMNHKGGADGTEMVNAVEVNRSNRNQEISGEYTIEAWTKFDFPGRGNTHSNFKWRWYHFDGTDWDQSRQLQNKIYKFRGTGKAWDWEVDIENGNYDYLMYADIDMDHPEVINELRNWGVWYTNTLNLDGFRIDAVKHIKYSYTRDWLTHVRNTTGKPMFAVAEFWKNDLAAIENYLNKTSWNHSVFDVPLHYNLYNASNSGGYFDMRNILNGSVVQKHPIHAVTFVDNHDSQPGEALESFVQSWFKPLAYALILTREQGYPSVFYGDYYGIPTHGVPSMKSKIDPLLQARQTYAYGTQHDYFDHHDIIGWTREGDSSHPNSGLATIMSDGPGGNKWMYVGKHKAGQVWRDITGNRSGTVTINADGWGNFTVNGGAVSVWVKQ

SEQ ID NO: 5 - DNA, 1458 bp, Bacillus sp. DSM12649
  CDS (1)..(1455): Codon-optimized oligonucleotide
catcataatggaacaaacggcacaatgatgcaatactttgaatggtatttaccgaacgatggaaaccattggaatagattacgcagcgatgcgtctaacctgaaagataaaggaatttcagctgtttggatcccgccggcatggaaaggagctagccaaaatgatgtgggctatggagcgtacgatctttacgatctgggagaatttaaccagaaaggcacaattcgcacaaaatatggcacaagaaatcaacttcaggcagcggtgaacgctttaaaatcaaatggaatccaagtctatggcgatgttgtgatgaatcataaaggcggagctgatgccacagaaatggtccgcgccgttgaagtgaacccgaataacagaaatcaggaagttagcggagaatacacaatcgaagcatggacaaaatttgattttccgggccgcggaaacacacattctaactttaaatggagatggtaccattttgatggcgttgattgggatcagtcaagaaaactgaacaaccgcatctacaaatttagaggcgatggaaaaggctgggattgggaagtggatacagaaaacggaaactacgattacctgatgtatgccgatatcgatatggatcatccggaagtcgttaacgaacttcgcaattggggcgtgtggtatacaaatacactgggacttgatggctttagaattgatgcggtcaaacatatcaaatatagctttacacgcgattggattaaccatgttagatctgcgacaggaaaaaatatgtttgcagtggcggaattttggaaaaatgatctgggcgctatcgaaaactaccttaacaaaacaaactggaaccattctgtctttgatgttccgctgcattacaacctttacaacgcgtcaaaaagcggcggaaactatgatatgcgccaaatttttaatggaacagtggtccagagacatccgatgcatgccgtgacatttgtcgataatcatgattctcaaccggaagaagcactggaatcatttgttgaagaatggtttaaaccgctggcttatgccttaacactgacacgcgaacagggatatccgagcgtgttttatggagattattatggcatcccgacacatggcgtcccggctatgaaatctaaaattgatccgatccttgaagctcgccaaaaatatgcctatggcagacagaacgattacctggatcatcataacatcatcggatggacaagagaaggcaacacagcacatccgaattcaggacttgcgacaattatgagcgatggcgctggcggaaacaaatggatgtttgtcggacgcaataaagcaggccaagtttggacagatattacaggaaacagagccggaacagtcacaatcaatgcagatggatggggcaactttagcgttaatggcggctcagtttcaatctgggtgaataaataa

SEQ ID NO: 6 - protein, 485 aa, Bacillus sp. DSM12649
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 7 - DNA, 1458 bp, Bacillus sp. SP722
  CDS (1)..(1455): Codon-optimized oligonucleotide
catcataatggcacaaacggaacaatgatgcaatattttgaatggcatctgccgaatgatggcaaccattggaatagactgcgcgatgatgcttctaaccttagaaatcgcggcattacagctatttggatcccgccggcctggaaaggaacatcacagaatgatgtcggctatggagcctatgatctgtatgatcttggcgaatttaaccaaaaaggaacagttcgcacaaaatatggaacaagatcacagttagaaagcgctatccatgccctgaaaaataacggcgttcaagtgtatggagatgttgtgatgaatcataaaggcggagcagatgcgacagaaaacgtgcttgctgtcgaagttaatccgaataaccgcaaccaggaaatttcaggcgattatacaatcgaagcctggacaaaatttgattttccgggccgcggaaacacatactcagattttaaatggagatggtaccattttgatggagtcgattgggatcaaagcagacaatttcagaaccgcatctacaaatttagaggcgatggaaaagcttgggattgggaagttgatagcgaaaatggcaactatgattatctgatgtatgccgatgtggatatggatcatccggaagtcgttaatgaacttagacgctggggcgaatggtacacaaacacactgaacctggatggatttagaatcgatgcggttaaacatatcaaatacagctttacacgcgattggttaacacatgttagaaatgcaacaggcaaagaaatgtttgctgtggccgaattttggaaaaacgatttaggagcgctggaaaactacctgaacaaaacaaactggaaccattctgtgtttgatgtcccgcttcattacaacctgtacaacgcatcaaacagcggcggaaattatgatatggcgaaactgcttaacggcacagtggtccaaaaacatccgatgcatgcagttacatttgtggataatcatgattctcaaccgggagaaagccttgaatcttttgtgcaggaatggtttaaaccgttagcatatgcgcttattttaacacgcgaacagggctatccgagcgtgttttatggcgattattatggaatcccgacacattctgtcccggcaatgaaagcgaaaattgatccgatccttgaagcacgccaaaattttgcgtatggaacacagcatgattactttgatcatcataacatcatcggctggacaagagaaggaaatacaacacatccgaactctggcttagcaacaattatgtcagatggaccgggcggagaaaaatggatgtatgtgggccaaaataaagcgggacaggtctggcatgatattacaggcaataaaccgggaacagtcacaatcaacgctgatggatgggccaattttagcgttaacggcggatctgtctcaatctgggttaaaagataa

SEQ ID NO: 8 - protein, 485 aa, Bacillus sp. SP722
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 9 - DNA, 1458 bp, Cytophaga sp.
  CDS (1)..(1455): Codon-optimized oligonucleotide
gccgccactaatgggacgatgatgcagtactttgaatggtacgttccgaatgatggccaacagtggaacaggctgagaacagacgcaccgtacttatcaagcgttggcattacggctgtctggacgccaccggcgtataagggtacttcacaagcggatgttggatacggaccatatgacttgtacgatttaggggagtttaaccagaaagggacagtacgcaccaaatatggaacaaaaggcgaactgaaaagcgccgtcaatacgctgcattccaatggaattcaggtctatggcgatgtggttatgaaccataaagcaggagcggactatactgagaatgttactgcagtggaagtgaatccatccaatcgaaaccaggaaacttcaggagaatacaacatacaggcgtggaccggctttaacttcccaggccgtggaacgacatatagcaactttaaatggcaatggtttcactttgatggtacagattgggatcaaagccgatcactctccaggatcttcaagtttcgcggtacaggcaaagcttgggattgggaggtgtcgtctgaaaacggcaattacgattaccttatgtatgcagatatcgactatgaccatcctgacgtcgttaatgagatgaagaaatggggagtatggtacgctaatgaagtaggtttagatgggtatcggttggatgccgtcaaacacatcaaattcagctttctcaaagattgggtcgataacgcgagagcagcaaccgggaaggagatgtttacagtgggtgaatattggcaaaatgacttaggagctcttaacaactatttggcgaaagtcaactataatcagtctctgtttgatgctccgcttcattacaacttctatgcggctagtacaggaggtgggtattatgacatgcgcaacattctgaataatacgctggttgcgtctaatcccacaaaagctgttaccctggtagaaaatcatgacacacaaccgggccagtctttggaatccacagtgcaaccgtggttcaaacctctcgcatatgcttttatcttaacacgctctggcggatatccctcagtgttctacggggatatgtatggcacaaagggaacgacgacacgggaaattcctgcactgaaatcgaagattgaacctcttcttaaggcacggaaagactatgcctatggcactcaacgtgattatattgacaaccctgacgtcattggctggacaagagagggagattcgaccaaagcgaaaagcggattagcaacggtaatcactgatggcccgggtggatctaagcgtatgtatgtcggtacgtcaaatgccggggaaatctggtacgatcttaccggcaatagaaccgataaaattacgataggcagtgatggttatgccacatttccggttaatggaggctcagtgagtgtatgggtgcaacagtaa

SEQ ID NO: 10 - protein, 485 aa, Cytophaga sp.
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRNQETSGEYNIQAWTGFNFPGRGTTYSNFKWQWFHFDGTDWDQSRSLSRIFKFRGTGKAWDWEVSSENGNYDYLMYADIDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGYYDMRNILNNTLVASNPTKAVTLVENHDTQPGQSLESTVQPWFKPLAYAFILTRSGGYPSVFYGDMYGTKGTTTREIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 11 - DNA, 36 bp, Artificial Sequence
  Synthetic oligonucleotide
gcagctcttgcagcagcgactacgaatggcacaatg

SEQ ID NO: 12 - DNA, 36 bp, Artificial Sequence
  Synthetic oligonucleotide
tagtttaatagattactgctggacccatactgagac

SEQ ID NO: 13 - DNA, 29 bp, Artificial Sequence
  Synthetic oligonucleotide
taatctattaaactagttatagggttatc

SEQ ID NO: 14 - DNA, 18 bp, Artificial Sequence
  Synthetic oligonucleotide
tgctgcaagagctgccgg

SEQ ID NO: 15 - DNA, 35 bp, Artificial Sequence
  Synthetic oligonucleotide
gcagctcttgcagcacatcataatggcacaaacgg

SEQ ID NO: 16 - DNA, 35 bp, Artificial Sequence
  Synthetic oligonucleotide
agtttaatagattactgtttcacccaaacagagac

SEQ ID NO: 17 - DNA, 38 bp, Artificial Sequence
  Synthetic oligonucleotide
gcagctcttgcagcacatcataatggaacaaacggcac

SEQ ID NO: 18 - DNA, 41 bp, Artificial Sequence
  Synthetic oligonucleotide
tagtttaatagattatttattcacccagattgaaactgagc

SEQ ID NO: 19 - DNA, 38 bp, Artificial Sequence
  Synthetic oligonucleotide
gcagctcttgcagcacatcataatggcacaaacggaac

SEQ ID NO: 20 - DNA, 41 bp, Artificial Sequence
  Synthetic oligonucleotide
tagtttaatagattatcttttaacccagattgagacagatc

SEQ ID NO: 21 - DNA, 32 bp, Artificial Sequence
  Synthetic oligonucleotide
gcagctcttgcagcagccgccactaatgggac

SEQ ID NO: 22 - DNA, 34 bp, Artificial Sequence
  Synthetic oligonucleotide
agtttaatagattactgttgcacccatacactca